(19) United States
(12) Patent Application Publication
(10) Pub. No.: US 2019/0353658 A1
(43) Pub. Date: Nov. 21, 2019

(54) Improved Methods Of Treating Lung Cancer Using Multiplex Proteomic Analysis

(71) Applicant: Expression Pathology, Inc., Rockville, MD (US)

(72) Inventors: Todd HEMBROUGH, Gaithersburg, MD (US); Fabiola CECCHI, Washington, DC (US); Jean-Charles SORIA, Villejuif (FR)

(21) Appl. No.: 16/463,746

(22) PCT Filed: Dec. 5, 2017
(86) PCT No.: PCT/US2017/064787

§ 371 Date: May 23, 2019

Related U.S. Application Data
(60) Provisional application No. 62/429,868, filed on Dec. 5, 2016.

(51) Int. Cl. G01N 33/574; G01N 33/68

(52) U.S. Cl. CPC G01N 33/57423; G01N 2800/52; G01N 33/6848

(57) ABSTRACT

The present invention provides methods for treating cancer patients comprising assaying tumor tissue from patients and identifying those patients most likely to respond to treatment with a platinum-based agent, such as cisplatin, in combination with pemetrexed. Methods are provided for identifying those lung cancer patients most likely to respond to treatment with the combination of cisplatin+pemetrexed chemotherapy agents (“CDDP+PEM”) by determining expression patterns of a set of 38 specific proteins directly in tumor cells derived from patient tumor tissue using SRM mass spectrometry. The method further comprising determining if the patient will respond to treatment with combination therapy, and when proteomic analysis of patient tissue indicates that the patient will respond to treatment with combination therapy, the patient is administered a regimen that includes the pemetrexed/platinum agent combination.

### CROSS-REFERENCE TO RELATED APPLICATIONS

This application claims priority to provisional application Ser. No. 62/429,868, filed Dec. 5, 2016, the contents of which are hereby incorporated by reference in their entirety.

### INTRODUCTION

Improved methods for treating cancer patients are provided by assaying tumor tissue from patients and identifying those patients most likely to respond to treatment with a platinum-based agent, such as cisplatin, in combination with pemetrexed. Cisplatin is a member of the platinum-based class of chemotherapeutic agents, while pemetrexed is a member of the antifolate class of drugs. More specifically, methods are provided for identifying those lung cancer patients most likely to respond to treatment with the combination of cisplatin+pemetrexed chemotherapy agents (“CDDP+PEM”) by determining expression patterns of a set of specific proteins directly in tumor cells derived from patient tumor tissue using SRM mass spectrometry. The 38 proteins that may be measured are shown in Table 1. Measurement of these proteins allows identification of proteomic signatures that allow selection of patients likely to profit from CDDP-PEM adjuvant therapy

Cisplatin (also known as cisplatinum, platamin, and neoplatin), is a member of a class of platinum-containing anti-cancer drugs, which also includes carboplatin and oxaliplatin. Once inside the cancer cell these platinum therapeutic agents bind to and cause crosslinking of DNA, which damages the DNA ultimately triggering apoptosis (programmed cell death) and death to cancer cells. Nucleotide excision repair (NER) is the primary DNA repair mechanism that removes the therapeutic platinum-DNA adducts from the tumor cell DNA. In the methods described herein, a “platinum-based agent” will be understood to include cisplatin, carboplatin and oxaliplatin. Similarly, reference to “cisplatin” will be understood to include other platinum-based chemotherapeutic agents unless indicated otherwise.

Pemetrexed, also known as Alimta, is chemically similar to folic acid and is a member of the class of folate antimetabolite chemotherapy drugs. It works by inhibiting three enzymes used in purine and pyrimidine synthesis-thymidylate synthase (TS), dihydrofolate reductase (DHFR), and glycinamide ribonucleotide formyltransferase (GARFT). By inhibiting the formation of precursor purine and pyrimidine nucleotides, pemetrexed prevents the formation of DNA and RNA, which are required for the growth and survival of both normal cells and cancer cells.

### BRIEF DESCRIPTION OF THE DRAWINGS

FIG. 1 shows a multiplex analysis that yields 3 prognostic subsets in the TASTE cohort (N=146)

FIG. 2 shows that patient subsets appear to have differences in Recurrence-free Survival (RFS).

### DETAILED DESCRIPTION

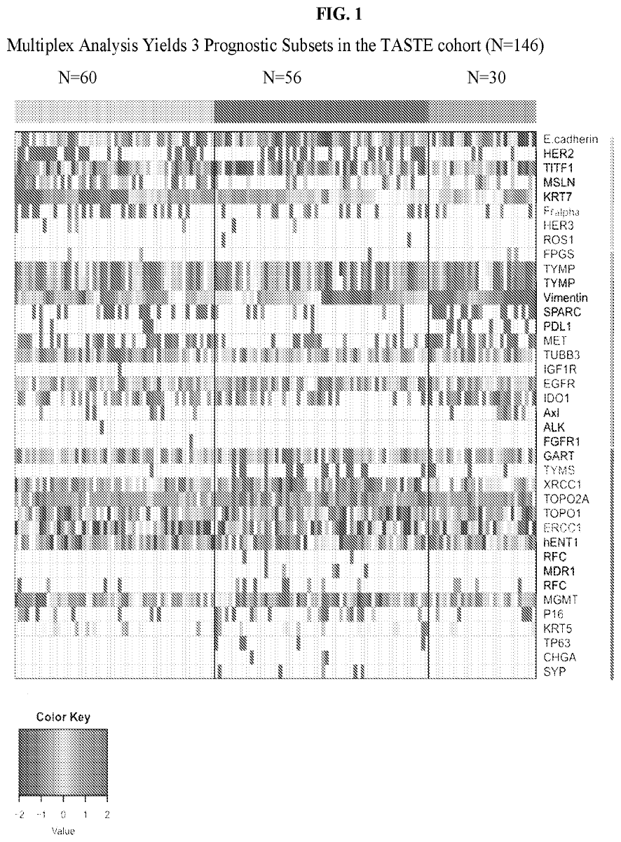

Using an SRM/MRM assay that simultaneously measures multiple protein biomarkers a correlation between biomarker expression and improved or reduced progression-free survival (PFS) was determined. The correlation is shown in FIG. 1. The results of the correlation allowed development of improved methods for treating lung cancer patients; more specifically the methods involve determining if a cancer patient, and specifically a NSCLC patient, will clinically respond in a favorable manner to combination therapy with pemetrexed and a platinum-based agent such as cisplatin.

The methods involve analyzing a tissue sample from the patient for expression of a collection of proteins comprising the proteins shown in Table 1 and the expression pattern of these proteins is used to guide the treatment regimen administered to the patient. More specifically, it has been found that expression in the patient tissue sample of three or more of a subgroup of the proteins shown in Table 1 is associated with a good clinical response to combination therapy with pemetrexed and a platinum-based agent, while expression of three or more proteins from a different subgroup of proteins is associated with a poor clinical response. Advantageously the sample is formalin-fixed tissue. When proteomic analysis of patient tissue indicates that the patient will respond to treatment with combination therapy with pemetrexed and a platinum-based agent, then that patient is treated with a regimen that includes the pemetrexed/platinum agent combination. For those patients where the analysis indicates that treatment with platinum agent plus pemetrexed is unlikely to be effective, an alternative therapeutic regimen may be used. Other therapeutics regimens include surgery (including wedge resection, segmental resection, lobectomy and pneumonectomy), radiation therapy, and targeted drug therapy (such as treatment with Afatinib (Gilotrif), Bevacizumab (Avastin), Ceritinib (Zykadia), Crizotinib (Xalkori), Erlotinib (Tarceva), Nivolumab (Opdivo) and Ramucirumab (Cyramza)).

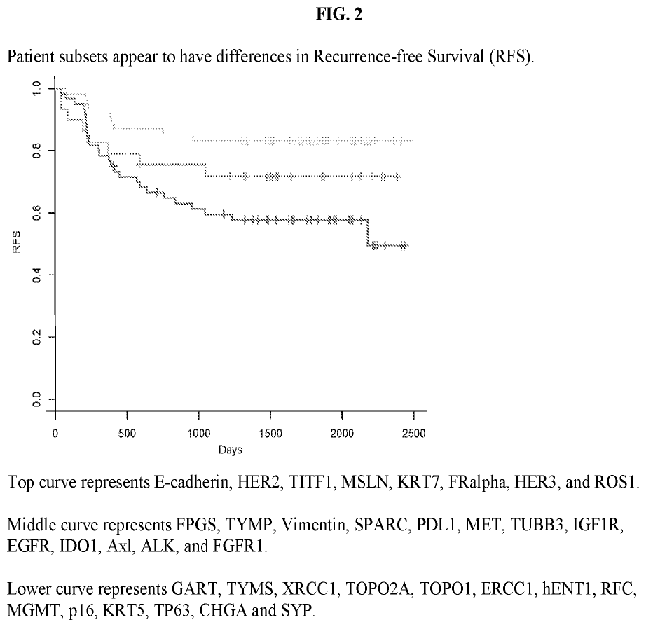

Patients who expressed one, two, or three or more or some or all of E-cadherin, HER2, TITF1, MSLN, KRT7, FRalpha, HER3, and ROS1 show improved PFS as shown in the top curve of FIG. 2 (p=0.004).

Patients who expressed one, two, three or more or some or all of GART, TYMS, XRCC1, TOPO2A, TOPO1, ERCC1, hENT1, RFC, MGMT, p16, KRT5, TP63, CHGA and SYP show poor PFS as shown in the bottom curve of FIG. 2.

Patients who expressed one or more or some or all of FPGS, TYMP, Vimentin, SPARC, PDL1, MET, TUBB3, IGF1R, EGFR, IDO1, Axl, ALK, and FGFR1 showed intermediate PFS as shown in the middle curve of FIG. 2.

An SRM/MRM assay can be used to measure peptide fragments from each of these protein directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue reagents and protocol available from Expression Pathology Inc. (Rockville, Md.).

The most widely and advantageously available form of tissue, and cancer tissue, from cancer patients is formalin fixed, paraffin embedded tissue. Formaldehyde/formalin fixation of surgically removed tissue is by far the most common method of preserving cancer tissue samples worldwide and is the accepted convention in standard pathology practice. Aqueous solutions of formaldehyde are referred to as formalin. “100%” formalin consists of a saturated solution of formaldehyde (this is about 40% by volume or 37% by mass) in water, with a small amount of stabilizer, usually methanol, to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods to analyze formalin fixed cancer tissue will be the most accepted and heavily utilized methods for analysis of cancer patient tissue.

Results from the SRM/MRM assay can be used to correlate accurate and precise quantitative levels of each of the proteins in Table 1 within the specific cancer of the patient from whom the tissue was collected and preserved, including lung cancer tissue. This not only provides diagnostic/prognostic information about the cancer, but also permits a physician or other medical professional to determine appropriate therapy for the patient. In this case, utilizing these assays can provide information about specific expression levels of the proteins in Table 1 expression simultaneously in cancer tissue and whether or not the patient from whom the cancer tissue was obtained will respond in a favorable way to combination therapy with pemetrexed and a platinum-based agent. Specific fragment peptides that can be used for detecting the proteins listed in Table 1 are shown in Table 2.

As described above, expression of three or more of the proteins E-cadherin, HER2, TITF1, MSLN, KRT7, FRalpha, HER3, and ROS1 is predictive of a favorable response to treatment with a combination of pemetrexed and a platinum-based agent as indicated by measurement of recurrence-free survival. Patients whose tumor tissue demonstrates this expression pattern advantageously are treated with a regimen including an effective amount of a platinum-based agent (such as cisplatin) and pemetrexed.

Various combinations of three of the proteins E-cadherin, HER2, TITF1, MSLN, KRT7, FRalpha, HER3, and ROS1 can be measured. For example, the following combinations may be measured:

E-cadherin, HER2, TITF1,

E-cadherin, HER2, MSLN

E-cadherin, HER2, KRT7

E-cadherin, HER2, FRalpha,

E-cadherin, HER2, HER3

E-cadherin, HER2, ROS1

E-cadherin, TITF1, MSLN,

E-cadherin, TITF1, KRT7

E-cadherin, TITF1, FRalpha

E-cadherin, TITF1, HER3

E-cadherin, TITF1, ROS1

E-cadherin, MSLN, KRT7

E-cadherin, MSLN, FRalpha

E-cadherin, MSLN, HER3

E-cadherin, MSLN ROS1

E-cadherin, KRT7, FRalpha

E-cadherin, KRT7, HER3

E-cadherin, KRT7, ROS1

E-cadherin, FRalpha, HER3

E-cadherin, FRalpha, ROS1

E-cadherin, HER3, and ROS1

HER2, TITF1, MSLN

HER2, TITF1, KRT7,

HER2, TITF1, FRalpha

HER2, TITF1, HER3

HER2, TITF1, ROS1

HER2, MSLN, KRT7

HER2, MSLN, FRalpha

HER2, MSLN, HER3

HER2, MSLN, ROS1

HER2, KRT7, FRalpha,

HER2, KRT7, HER3

HER2, KRT7, ROS1

TITF1, MSLN, KRT7,

TITF1, MSLN, FRalpha

TITF1, MSLN, HER3

TITF1, MSLN, ROS1

TITF1, KRT7, FRalpha,

TITF1, KRT7, HER3,

TITF1, KRT7 ROS1

MSLN, KRT7, FRalpha

MSLN, KRT7, HER3

MSLN, KRT7, ROS1

KRT7, FRalpha, HER3

KRT7, FRalpha, ROS1

KRT7, HER3, ROS1

FRalpha, HER3, ROS1

Similarly, various combinations of four of the proteins E-cadherin, HER2, TITF1, MSLN, KRT7, FRalpha, HER3, and ROS1 can be measured. For example, the following combinations may be measured:

E-cadherin, HER2, TITF1, MSLN,

E-cadherin, HER2, TITF1, KRT7

E-cadherin, HER2, TITF1, FRalpha

E-cadherin, HER2, TITF1, HER3

E-cadherin, HER2, TITF1, ROS1

E-cadherin, TITF1, MSLN, KRT7,

E-cadherin, TITF1, MSLN, FRalpha

E-cadherin, TITF1, MSLN, HER3

E-cadherin, TITF1, MSLN, ROS1

E-cadherin, MSLN, KRT7, FRalpha

E-cadherin, MSLN, KRT7, HER3

E-cadherin, MSLN, KRT7, ROS1

E-cadherin, KRT7, FRalpha, HER3

E-cadherin, KRT7, FRalpha ROS1

E-cadherin, FRalpha, HER3, and ROS1

HER2, TITF1, MSLN, KRT7,

HER2, TITF1, MSLN, FRalpha

HER2, TITF1, MSLN, HER3

HER2, TITF1, MSLN, ROS1

HER2, MSLN, KRT7, FRalpha

HER2, MSLN, KRT7, HER3

HER2, MSLN, KRT7, ROS1

HER2, KRT7, FRalpha, HER3

HER2, KRT7, FRalpha, ROS1

HER2, FRalpha, HER3, and ROS1

TITF1, MSLN, KRT7, FRalpha

TITF1, MSLN, KRT7, HER3,

TITF1, MSLN, KRT7, ROS1

MSLN, KRT7, FRalpha, HER3, ROS1

MSLN, KRT7, FRalpha, ROS1

KRT7, FRalpha, HER3 ROS1

Similarly, various combinations of five of the proteins E-cadherin, HER2, TITF1, MSLN, KRT7, FRalpha, HER3, and ROS1 can be measured. For example, the following combinations may be measured:

E-cadherin, HER2, TITF1, MSLN, KRT7

E-cadherin, HER2, TITF1, MSLN, FRalpha

E-cadherin, HER2, TITF1, MSLN, HER3

E-cadherin, HER2, TITF1, MSLN, ROS1

E-cadherin, TITF1, MSLN, KRT7, FRalpha

E-cadherin, TITF1, MSLN, KRT7, HER3

E-cadherin, TITF1, MSLN, KRT7, ROS1

E-cadherin, MSLN, KRT7, FRalpha, HER3

E-cadherin, MSLN, KRT7, FRalpha, ROS1

E-cadherin, KRT7, FRalpha, HER3, and ROS1

HER2, TITF1, MSLN, KRT7, FRalpha,

HER2, TITF1, MSLN, KRT7, HER3

HER2, TITF1, MSLN, KRT7, ROS1

TITF1, MSLN, KRT7, FRalpha, HER3

TITF1, MSLN, KRT7, FRalpha, ROS1

MSLN, KRT7, FRalpha, HER3, ROS1

Similarly, various combinations of six of the proteins E-cadherin, HER2, TITF1, MSLN, KRT7, FRalpha, HER3, and ROS1 can be measured. For example, the following combinations may be measured:

E-cadherin, HER2, TITF1, MSLN, KRT7, FRalpha,

E-cadherin, HER2, TITF1, MSLN, KRT7, HER3

E-cadherin, HER2, TITF1, MSLN, KRT7, ROS1

E-cadherin, TITF1, MSLN, KRT7, FRalpha, HER3

E-cadherin, TITF1, MSLN, KRT7, FRalpha, ROS1

E-cadherin, MSLN, KRT7, FRalpha, HER3, ROS1

HER2, TITF1, MSLN, KRT7, FRalpha, HER3

HER2, TITF1, MSLN, KRT7, FRalpha, ROS1

HER2, TITF1, KRT7, FRalpha, HER3, ROS1

HER2, TITF1, MSLN, FRalpha, HER3, ROS1

HER2, TITF1, MSLN, KRT7, HER3, ROS1

HER2, TITF1, MSLN, FRalpha, HER3, ROS1

TITF1, MSLN, KRT7, FRalpha, HER3, ROS1

Similarly, various combinations of seven of the proteins E-cadherin, HER2, TITF1, MSLN, KRT7, FRalpha, HER3, and ROS1 can be measured. For example, the following combinations may be measured:

HER2, TITF1, MSLN, KRT7, FRalpha, HER3, and ROS1

E-cadherin, TITF1, MSLN, KRT7, FRalpha, HER3, and ROS1

E-cadherin, HER2, MSLN, KRT7, FRalpha, HER3, and ROS1

E-cadherin, HER2, TITF1, KRT7, FRalpha, HER3, and ROS1

E-cadherin, HER2, TITF1, MSLN, FRalpha, HER3, and ROS1

E-cadherin, HER2, TITF1, MSLN, KRT7, HER3, and ROS1

E-cadherin, HER2, TITF1, MSLN, KRT7, FRalpha, and ROS1

E-cadherin, HER2, TITF1, MSLN, KRT7, FRalpha, HER3.

Expression of three or more, four or more, five or more, six or more, seven or more, eight or more, nine or more, ten or more, eleven or more, twelve or more, and thirteen or more, of the proteins GART, TYMS, XRCC1, TOPO2A, TOPO1, ERCC1, hENT1, RFC, MGMT, p16, KRT5, TP63, CHGA and SYP can be measured in all possible combinations as shown above.

Presently the most widely-used and applied methodology to determine protein presence in cancer patient tissue, especially FFPE tissue, is immunohistochemistry (IHC). IHC methodology utilizes an antibody to detect the protein of interest. The results of an IHC test are most often interpreted by a pathologist or histotechnologist. This interpretation is subjective and does not provide quantitative data that are predictive of sensitivity to therapeutic agents that target specific oncoprotein targets, such as cisplatin/pemetrexed sensitivity in a tumor cell population.

Research from other IHC assays, such as the Her2 IHC test suggest the results obtained from such tests may be wrong. This is probably because different labs have different rules for classifying positive and negative IHC status. Each pathologist running the tests also may use different criteria to decide whether the results are positive or negative. In most cases, this happens when the test results are borderline, meaning that the results are neither strongly positive nor strongly negative. In other cases, tissue from one area of cancer tissue can test positive while tissue from a different area of the cancer tests negative. Inaccurate IHC test results may mean that patients diagnosed with cancer do not receive the best possible care. If all or part of a cancer is positive for a specific target oncoprotein but test results classify it as negative, physicians are unlikely to recommend the correct therapeutic treatment, even though the patient could potentially benefit from those agents. If a cancer is oncoprotein target negative but test results classify it as positive, physicians may recommend a specific therapeutic treatment, even though the patient is unlikely to get any benefits and is exposed to the agent's secondary risks.

Thus there is great clinical value in the ability to correctly measure expression levels of the proteins listed in Table 1 in tumors, especially lung tumors, so that the patient will have the greatest chance of receiving the most optimal treatment.

Detection of peptides and determining quantitative levels of the proteins in Table 1 may be carried out in a mass spectrometer by the SRM/MRM methodology, whereby the SRM/MRM signature chromatographic peak area of each peptide is determined within a complex peptide mixture present in a Liquid Tissue lysate (see U.S. Pat. No. 7,473,532, as described above). Quantitative levels of the proteins are then measured by the SRM/MRM methodology whereby the SRM/MRM signature chromatographic peak area of an individual specified peptide from each of the proteins in one biological sample is compared to the SRM/MRM signature chromatographic peak area of a known amount of a “spiked” internal standard for each of the individual specified fragment peptides. In one embodiment, the internal standard is a synthetic version of the same exact fragment peptides where the synthetic peptides contain one or more amino acid residues labeled with one or more heavy isotopes. Such isotope labeled internal standards are synthesized so that mass spectrometry analysis generates a predictable and consistent SRM/MRM signature chromatographic peak that is different and distinct from the native fragment peptide chromatographic signature peaks and which can be used as comparator peaks. Thus when the internal standard is spiked in known amounts into a protein or peptide preparation from a biological sample and analyzed by mass spectrometry, the SRM/MRM signature chromatographic peak area of the native peptide is compared to the SRM/MRM signature chromatographic peak area of the internal standard peptide, and this numerical comparison indicates either the absolute molarity and/or absolute weight of the native peptide present in the original protein preparation from the biological sample. Quantitative data for fragment peptides are displayed according to the amount of protein analyzed per sample.

In order to develop the SRM/MRM assay for the fragment peptides additional information beyond simply the peptide sequence needs to be utilized by the mass spectrometer. That additional information is important in directing and instructing the mass spectrometer, (e.g., a triple quadrupole mass spectrometer) to perform the correct and focused analysis of the specified fragment peptides. An important consideration when conducting an SRM/MRM assay is that such an assay may be effectively performed on a triple quadrupole mass spectrometer. That type of a mass spectrometer may be considered to be presently the most suitable instrument for analyzing a single isolated target peptide within a very complex protein lysate that may consist of hundreds of thousands to millions of individual peptides from all the proteins contained within a cell. The additional information provides the triple quadrupole mass spectrometer with the correct directives to allow analysis of a single isolated target peptide within a very complex protein lysate that may consist of hundreds of thousands to millions of individual peptides from all the proteins contained within a cell. Although SRM/MRM assays can be developed and performed on any type of mass spectrometer, including a MALDI, ion trap, ion trap/quadrupole hybrid, or triple quadrupole, presently the most advantageous instrument platform for SRM/MRM assay is often considered to be a triple quadrupole instrument platform. The additional information about target peptides in general, and in particular about the specified fragment peptides for the proteins in Table 1, may include one or more of the mono isotopic mass of each peptide, its precursor charge state, the precursor m/z value, the m/z transition ions, and the ion type of each transition ion.

Proteomic Analysis of Tumor Tissue

Tumor samples were obtained from a cohort of patients suffering from cancer, in this case lung cancer. The lung tumor samples were formalin-fixed using standard methods and the level of the proteins shown in Table 1 in the samples was measured using the methods as described above. The tissue samples optionally may also be examined using IHC and FISH using methods that are well known in the art. The patients in the cohort were treated with a combination of cisplatin and pemetrexed therapeutic agents and the response of the patients was measured using methods that are well known in the art, for example by recording the overall survival of the patients at time intervals after treatment. Expression levels of the proteins of Table 1 were correlated with PFS using statistical methods that are well known in the art, for example by determining the lowest p value of a log rank test. This analysis was used to identify those patients whose protein expression profiles indicate that they may likely benefit from the combination of the combination cisplatin/pemetrexed therapeutic regimen. The skilled artisan will recognize that cisplatin/pemetrexed is the most common treatment regimen for NSCLC patients.

Because both nucleic acids and protein can be analyzed from the same Liquid Tissue biomolecular preparation it is possible to generate additional information about disease diagnosis and drug treatment decisions from the nucleic acids in same sample upon which proteins were analyzed. For example, if the proteins shown in Table 1 proteins are expressed by certain cells at increased levels, when assayed by SRM the data can provide information about the state of the cells and their potential for uncontrolled growth, choice of optimal therapy, and potential drug resistance. At the same time, information about the status of genes and/or the nucleic acids and proteins they encode (e.g., mRNA molecules and their expression levels or splice variations) can be obtained from nucleic acids present in the same Liquid Tissue™ biomolecular preparation. Nucleic acids can be assessed simultaneously to the SRM analysis of proteins, including the proteins of Table 1. In one embodiment, information about the Table 1 proteins and/or one, two, three, four or more additional proteins may be assessed by examining the nucleic acids encoding those proteins. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

TABLE 1

| | |
|---|---|
| E-cadherin | |
| HER2 | Human epidermal growth factor receptor 2 |
| TITF1 | Thyroid transcription Factor 1 |
| MSLN | Mesothelin |
| KRT7 | Keratin 7 |
| FRalpha | Folate receptor alpha |
| HER3 | Human epidermal growth factor receptor 3 |
| ROS1 | gene product of the Ros1 gene |
| FPGS | Folylpolyglutamate Synthase |
| TYMP | thymidine phosphorylase |
| Vimentin | |
| SPARC | secreted protein acidic and rich in cysteine |
| PDL1 | Programmed death-ligand 1 |
| MET | gene product of met gene |
| TUBB3 | tubulin beta 3 |
| IGF1R | Insulin-like growth factor 1 receptor |
| EGFR | Epidermal growth factor receptor |
| IDO1 | Indoleamine 2,3-Dioxygenase 1 |
| Axl | |
| ALK | Anaplastic lymphoma kinas |
| FGFR1 | Fibroblast growth factor 1 |
| GART | Phosphoribosylglycinamide Formyltransferase |
| TYMS | Thymidylate synthase |
| XRCC1 | X-ray repair cross-complementing protein 1 |
| TOPO2A | Topoisomerase 2A |
| TOPO1 | Topoisomerase 1 |
| ERCC1 | DNA excision repair protein |
| hENT1 | human equilibrative nucleoside transporter 1 |
| RFC | Replication factor C, |
| MGMT | O-6-methylguanine-DNA methyltransferase |
| p16 | cyclin-dependent kinase inhibitor 2A |
| KRT5 | Keratin 5 |
| TP63 | transformation-related protein 63 |
| CHGA | Chromogranin A |
| SYP | Synaptophysin |

TABLE 2

| SEQ ID NO | Protein | Peptide Sequence |
|---|---|---|
| SEQ ID NO 1 | E-Cadherin | NTGVISVVTTGLDR |
| SEQ ID NO 2 | HER2 | ELVSEFSR |
| SEQ ID NO 3 | TITF1 | FPAISR |
| SEQ ID NO 4 | MSLN | GSLLSEADVR |
| SEQ ID NO 5 | KRT7 | LPDIFEAQIAGLR |
| SEQ ID NO 6 | FRalpha | DVSYLYR |
| SEQ ID NO 7 | HER3 | LAEVPDLLEK |
| SEQ ID NO 8 | ROS1 | GEGLLPVR |
| SEQ ID NO 9 | FPGS | TGFFSSPHLVQVR |
| SEQ ID NO 10 | TYMP | DGPALSGPQSR |
| SEQ ID NO 11 | Vimentin | SLYASSPGGVYATR |
| SEQ ID NO 12 | SPARC | NVLVTLYER |
| SEQ ID NO 13 | PDL1 | LQDAGVYR |
| SEQ ID NO 14 | MET | TEFTTALQR |
| SEQ ID NO 15 | TUBB3 | ISVYYNEASSHK |
| SEQ ID NO 16 | IGF1R | GNLLINIR |
| SEQ ID NO 17 | EGFR | IPLENLQIIR |
| SEQ ID NO 18 | IDO1 | HLPDLIESGQLR |
| SEQ ID NO 19 | AXL | APLQGTLLGYR |
| SEQ ID NO 20 | ALK | DPEGVPPLLVSQQAK |
| SEQ ID NO 21 | FGFR1 | IGPDNLPYVQILK |
| SEQ ID NO 22 | GART | VLAVTAIR |
| SEQ ID NO 23 | TYMS | EEGDLGPVYGFQWR |
| SEQ ID NO 24 | XRCC1 | TPATAPVPAR |
| SEQ ID NO 25 | TOPO2A | TLAVSGLGVVGR |
| SEQ ID NO 26 | TOPO1 | AEEVATFFAK |
| SEQ ID NO 27 | ERCC1 | EGVPQPSGPPAR |
| SEQ ID NO 28 | hENT1 | WLPSLVLAR |
| SEQ ID NO 29 | RFC | AAQALSVQDK |
| SEQ ID NO 30 | MGMT | TTLDSPLGK |
| SEQ ID NO 31 | p16 | ALLEAGALPNAPNSYGR |
| SEQ ID NO 32 | KRT5 | ISISTSGGSFR |
| SEQ ID NO 33 | TP63 | TPSSASTVSVGSSETR |
| SEQ ID NO 34 | CHGA | VAHQLQALR |
| SEQ ID NO 35 | SYP | ETGWAAPFLR |

1. A method of treating a patient suffering from lung cancer comprising:
(a) measuring the expression of a set of proteins in a sample of tumor tissue obtained from the patient, wherein said set of proteins comprises E-cadherin, HER2, TITF1, MSLN, KRT7, FRalpha, HER3, ROS1, GART, TYMS, XRCC1, TOPO2A, TOPO1, ERCC1, hENT1, RFC, MGMT, p16, KRT5, TP63, CHGA and SYP;
(b) treating the patient with a therapeutic regimen comprising an effective amount of a platinum-based agent and pemetrexed when expression of at least three proteins selected from the group consisting of E-cadherin, HER2, TITF1, MSLN, KRT7, FRalpha, HER3, ROS1, is detected, and
(c) treating the patient with a therapeutic regimen that does not comprise an effective amount of a platinum-based agent and pemetrexed when expression of at least three proteins selected from the group consisting of GART, TYMS, XRCC1, TOPO2A, TOPO1, ERCC1, hENT1, RFC, MGMT, p16, KRT5, TP63, CHGA and SYP is detected.

2. The method according to claim 1 wherein at least four, at least five, at least six, at least seven, or all eight proteins selected from the group consisting of E-cadherin, HER2, TITF1, MSLN, KRT7, FRalpha, HER3, ROS1, is detected.

3. The method according to claim 1 wherein at least four, at least five, at least six, at least seven, at least eight, at least nine, at least ten, at least eleven, at least twelve, at least thirteen or all fourteen proteins selected from the group consisting of GART, TYMS, XRCC1, TOPO2A, TOPO1, ERCC1, hENT1, RFC, MGMT, p16, KRT5, TP63, CHGA and SYP is detected.

4. The method according to claim 1, wherein said proteins are detected by mass spectrometric detection of a fragment peptide in a protein digest prepared from said sample of tumor tissue.

5. The method according to claim 4, wherein said protein digest comprises a protease digest.

6. The method according to claim 5, wherein said protein digest comprises a trypsin digest.

7. The method according to claim 4, wherein said mass spectrometric detection comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

8. The method according to claim 7, wherein a mode of mass spectrometry used is Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

9. The method according to claim 4, wherein said fragment peptide is selected from the group consisting of the peptides of SEQ ID NOs 1-8 and SEQ ID NOs 22-35.

10. The method according to claim 1, wherein the sample of tumor tissue is a cell, collection of cells, or a solid tissue.

11. The method of claim 10, wherein the tumor sample is formalin fixed solid tissue.

12. The method of claim 11, wherein the tissue is paraffin embedded tissue.


SEQUENCE LISTING (35 sequences)

SEQ ID NO: 1 - protein, 14 aa, Homo sapiens
NTGVISVVTTGLDR

SEQ ID NO: 2 - protein, 8 aa, Homo sapiens
ELVSEFSR

SEQ ID NO: 3 - protein, 6 aa, Homo sapiens
FPAISR

SEQ ID NO: 4 - protein, 10 aa, Homo sapiens
GSLLSEADVR

SEQ ID NO: 5 - protein, 13 aa, Homo sapiens
LPDIFEAQIAGLR

SEQ ID NO: 6 - protein, 7 aa, Homo sapiens
DVSYLYR

SEQ ID NO: 7 - protein, 10 aa, Homo sapiens
LAEVPDLLEK

SEQ ID NO: 8 - protein, 8 aa, Homo sapiens
GEGLLPVR

SEQ ID NO: 9 - protein, 13 aa, Homo sapiens
TGFFSSPHLVQVR

SEQ ID NO: 10 - protein, 11 aa, Homo sapiens
DGPALSGPQSR

SEQ ID NO: 11 - protein, 14 aa, Homo sapiens
SLYASSPGGVYATR

SEQ ID NO: 12 - protein, 9 aa, Homo sapiens
NVLVTLYER

SEQ ID NO: 13 - protein, 8 aa, Homo sapiens
LQDAGVYR

SEQ ID NO: 14 - protein, 9 aa, Homo sapiens
TEFTTALQR

SEQ ID NO: 15 - protein, 12 aa, Homo sapiens
ISVYYNEASSHK

SEQ ID NO: 16 - protein, 8 aa, Homo sapiens
GNLLINIR

SEQ ID NO: 17 - protein, 10 aa, Homo sapiens
IPLENLQIIR

SEQ ID NO: 18 - protein, 12 aa, Homo sapiens
HLPDLIESGQLR

SEQ ID NO: 19 - protein, 11 aa, Homo sapiens
APLQGTLLGYR

SEQ ID NO: 20 - protein, 15 aa, Homo sapiens
DPEGVPPLLVSQQAK

SEQ ID NO: 21 - protein, 13 aa, Homo sapiens
IGPDNLPYVQILK

SEQ ID NO: 22 - protein, 8 aa, Homo sapiens
VLAVTAIR

SEQ ID NO: 23 - protein, 14 aa, Homo sapiens
EEGDLGPVYGFQWR

SEQ ID NO: 24 - protein, 10 aa, Homo sapiens
TPATAPVPAR

SEQ ID NO: 25 - protein, 12 aa, Homo sapiens
TLAVSGLGVVGR

SEQ ID NO: 26 - protein, 10 aa, Homo sapiens
AEEVATFFAK

SEQ ID NO: 27 - protein, 12 aa, Homo sapiens
EGVPQPSGPPAR

SEQ ID NO: 28 - protein, 9 aa, Homo sapiens
WLPSLVLAR

SEQ ID NO: 29 - protein, 10 aa, Homo sapiens
AAQALSVQDK

SEQ ID NO: 30 - protein, 9 aa, Homo sapiens
TTLDSPLGK

SEQ ID NO: 31 - protein, 17 aa, Homo sapiens
ALLEAGALPNAPNSYGR

SEQ ID NO: 32 - protein, 11 aa, Homo sapiens
ISISTSGGSFR

SEQ ID NO: 33 - protein, 16 aa, Homo sapiens
TPSSASTVSVGSSETR

SEQ ID NO: 34 - protein, 9 aa, Homo sapiens
VAHQLQALR

SEQ ID NO: 35 - protein, 10 aa, Homo sapiens
ETGWAAPFLR